(19) United States
(12) Patent Application Publication
(10) Pub. No.: US 2008/0255355 A1
(43) Pub. Date: Oct. 16, 2008

(54) Chiral Diphosphonites as Ligands in the Ruthenium-Catalyzed Enantioselective Reduction of Ketones, B-Ketoesters and Ketimines

(75) Inventors: Manfred T. Reetz, Mulheim an der Ruhr (DE); Xiaoguang Li, New York, NY (US)

Correspondence Address: NORRIS, MCLAUGHLIN & MARCUS, PA, 875 THIRD AVENUE, 18TH FLOOR, NEW YORK, NY, 10022, US

(73) Assignee: STUDIENGESELLSCHAFT KOHLE MBH, Mulheim an der Ruhr (DE)

(21) Appl. No.: 11/915,924

(22) PCT Filed: May 30, 2006
(86) PCT No.: PCT/DE2006/000929

§ 371 Date: Nov. 29, 2007

(30) Foreign Application Priority Data
Jun. 2, 2005 (DE) 10 2005 025 797.6

(51) Int. Cl. C07F 15/00; C07F 9/6571; C07C 37/00

(52) U.S. Cl. 544/225; 556/136; 549/206; 556/9; 549/3; 556/21; 546/2; 568/700

(57) ABSTRACT

Chiral ruthenium complexes are disclosed, obtained by reaction of a ruthenium salt with a chiral diphosphonite. Chiral diols with the general structure given in scheme 1 are preferably used as chiral diphosphonites. Said ruthenium complexes can be simply and economically obtained and provide high enantioselectivity on reduction of ketones, β-ketoesters and ketimines.

The present invention relates to the preparation of ruthenium complexes of chiral diphosphonites and to their use as catalysts in the asymmetric reduction of ketones, β-keto esters and ketimines, the products being enantiomerically pure or enriched alcohols or amines which constitute industrially valuable units in the preparation of compounds such as pharmaceuticals, crop protection compositions, fragrances and natural products, or intermediates in their syntheses.

The transition metal-catalyzed enantioselective reduction of prochiral ketones I, β-keto esters III and ketimines V requires enantiomerically pure or enriched chiral alcohols II, β-hydroxyesters IV or amines VI, which are valuable intermediates for the industrial preparation of a multitude of active pharmaceutical ingredients, crop protection compositions, fragrances or other products (R. Noyori, Angew. Chem. Int. Ed. 2002, 41, 2008-2022; H.-U. Blaser, C. Malan, B. Pugin, F. Spindler, H. Steiner, M. Studer, Adv. Synth. Catal. 2003, 345, 103-151; M. J. Palmer, M. Wills, Tetrahedron: Asymmetry 1999, 10, 2045-2061). A multitude of catalyst systems has been developed for such reductions, either by H₂ hydrogenation or by transfer hydrogenation, for example using isopropanol as a hydrogen donor. Some less well known chiral ligands give rise to high enantioselectivities (ee>90%) for some but not all substrates (R. Noyori, Angew. Chem. Int. Ed. 2002, 41, 2008-2022; H.-U. Blaser, C. Malan, B. Pugin, F. Spindler, H. Steiner, M. Studer, Adv. Synth. Catal. 2003, 345, 103-151; M. J. Palmer, M. Wills, Tetrahedron: Asymmetry 1999, 10, 2045-2061).

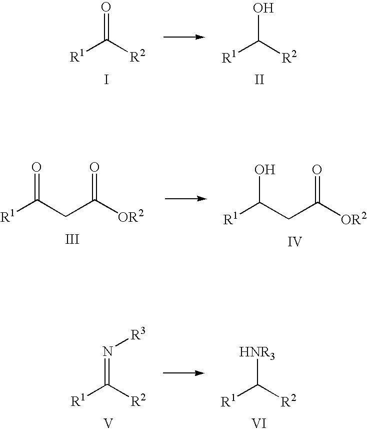

Disadvantages of the common methods are the high costs of a multistage preparation for the chiral ligands which are required to obtain a high ee, and the only limited general applicability; for example, many ketones of interest lead to alcohols with low enantioselectivity. For example, the Ru catalyst comprises with optically active BINAP and a chiral diamine two expensive ligands (R. Noyori, Angew. Chem. Int. Ed. 2002, 41, 2008-2022). In one of the currently most effective processes for asymmetric ketone reduction, which has also been described by Noyori, Ru(II) complexed by an aromatic compound and a monotosylated chiral diamine is used, the complexes acting as catalysts in transfer hydrogenation with isopropanol as a hydrogen donor under basic conditions (R. Noyori, S. Hashiguchi, Acc. Chem. Res. 1997, 30, 97-102). The disadvantages of this catalyst system lie in the laborious preparation of the chiral tosylated diamine ligands and the fact that generally only aryl alkyl ketones (I where R¹=aryl and R²=alkyl) react with high enantioselectivity (ee>90%), while many alkyl alkyl ketones (I where R¹=alkyl and R²=a different alkyl) lead to only moderate or low enantioselectivities. For example, the best Noyori catalyst reduces methyl cyclohexyl ketone (I, R¹═CH₃; R²=c-C₅H₁₁) with an ee of only 60% (J. Takehara, S. Hashiguchi, A. Fujii, S.-I. Inoue, T. Ikariya, R. Noyori, Chem. Commun. (Cambridge, U.K.) 1996, 233-234). This catalyst system was improved with regard to the enantioselectivity using appropriate Ru(II) complexes in which the aromatic ligand and the chiral tosylated diamine ligands are bonded to one another covalently through an ether, which, though, makes the synthesis of the ligand system much more complicated and expensive (A. M. Hayes, D. J. Morris, G. J. Clarkson, M. Wills, J. Am. Chem. Soc. 2005, 127, 7318-7319). Furthermore, even the enantioselectivity for alkyl alkyl ketones such as methyl cyclohexyl ketone (I, R¹═CH₃; R²=c-C₆H₁₁) is improved only slightly (ee=69%) (A. M. Hayes, D. J. Morris, G. J. Clarkson, M. Wills, J. Am. Chem. Soc. 2005, 127, 7318-7319).

The present invention eliminates many of the above-described disadvantages.

The present invention provides chiral ruthenium complexes which can be obtained by reacting one or more ruthenium salts with a chiral diphosphonite.

The invention further provides a process for enantioselective reduction of prochiral ketones, β-keto esters and ketimines using these ruthenium complexes as catalysts in H₂ hydrogenation or transfer hydrogenation.

The invention utilizes ruthenium complexes with inexpensive chiral diphosphonites obtainable in a simple manner. Phosphonites are compounds having a carbon-phosphorus bond and two phosphorus-oxygen bonds. Nitrogen analogs, i.e. derivatives of the phosphonites in which one or both oxygen radicals have been replaced by an amino group are likewise encompassed by the present invention. The ligands of the present invention consist of an achiral or chiral backbone to which two phosphonite radicals are bonded, where each radical contains a chiral ligand such as a chiral diol (Scheme 1), diamine (Scheme 2) or an amino alcohol (Scheme 3), all stereoisomeric forms also being part of the invention:

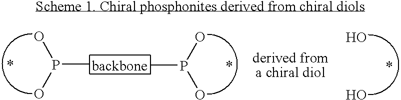

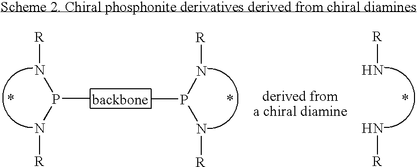

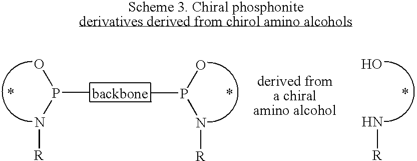

Many of these diphosphonites and their nitrogen analogs have already been described in the literature (M. T. Reetz, A. Gosberg, R. Goddard, S.-H. Kyung, Chem. Commun. (Cambridge, U.K.) 1998, 2077-2078; I. E. Nifant'ev, L. F. Manzhukova, M. Y. Antipin, Y. T. Struchkov, E. E. Nifant'ev, Zh. Obshch. Khim. 1995, 65, 756-760; J. I. van der Vlugt, J. M. J. Paulusse, E. J. Zijp, J. A. Tijmensen, A. M. Mills, A. L. Spek, C. Clayer, D. Vogt, Eur. J. Inorg. Chem. 2004, 4193-4201; M. T. Reetz, A. Gosberg, Int. Pat. Appl. WO 00/14096, 2000), but none of the compounds described have been used as a ligand in Ru(II)-catalyzed reactions. The present invention also encompasses the preparation of these novel Ru(II) complexes and their use as catalysts in the asymmetric reduction of ketones, β-keto esters and imines.

It is known that the type of backbone in the diphosphonites can vary considerably, which enables a structural variety in the preparation of the corresponding Ru(II) complexes. Simple alkyl or substituted alkyl chains, i.e. —(CH₂)ₙ— where n=1, 2, 3, 4, 5, 6, 7 or 8, may serve as a backbone, as may alkyl chains which contain heteroatoms in the chain, e.g. —CH₂CH₂CH₂OCH₂CH₂CH₂—, but also aromatic radicals such as o,o-disubstituted benzene derivatives. One example of a chiral backbone is the trans-1,2-disubstituted cyclopentane derivative. Excluded as backbones are ferrocene derivatives which have a phosphorus radical on every cyclopentadienyl group (I. E. Nifant'ev, L. F. Manzhukova, M. Y. Antipin, Y. T. Struchkov, E. E. Nifant'ev, Zh. Obshch. Khim. 1995, 65, 756-760; M. T. Reetz, A. Gosberg, R. Goddard, S.-H. Kyung, Chem. Commun. (Cambridge, U.K.) 1998, 2077-2078; M. T. Reetz, A. Gosberg, Int. Pat. Appl. WO 00/14096, 2000). A particularly inexpensive chiral assistant on the phosphorus in the diphosphonites is, as well as many other possibilities, (R)- or (S)-dinaphthol (BINOL). Typical examples are shown below (VII-X):

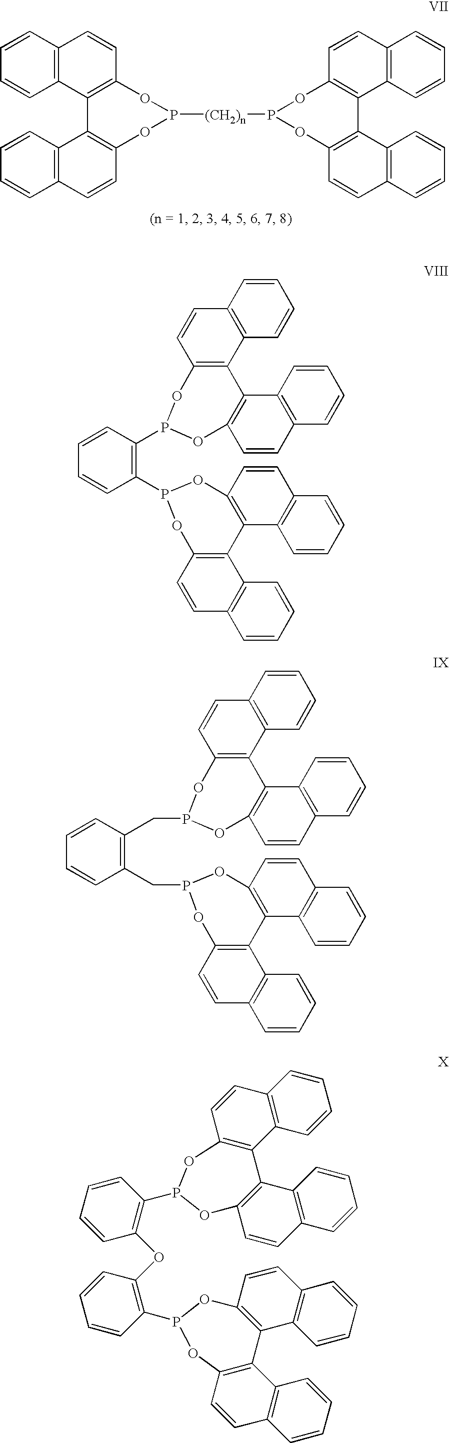

In addition, typical examples can also be prepared from the derivatives of xanthene (e.g. XI or XII), homoxanthene (e.g. XIII), sexanthene (e.g. XIV), thixanthene (e.g. XV), nixanthene (e.g. XVI), phosxanthene (e.g. XVII), benzoxanthene (e.g. XVIII), acridine (e.g. XIX) or dibenzofuran (e.g. XX):

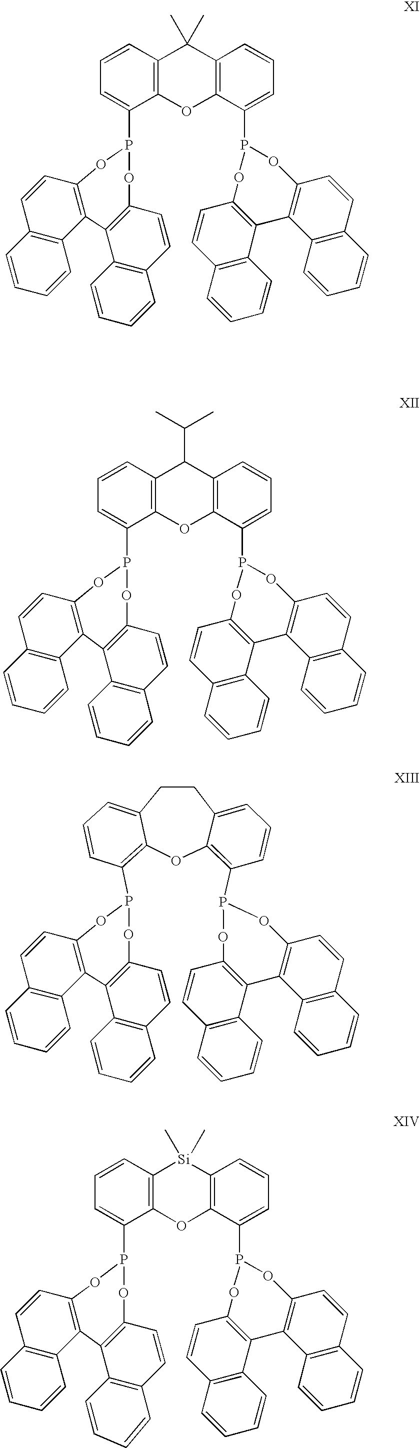

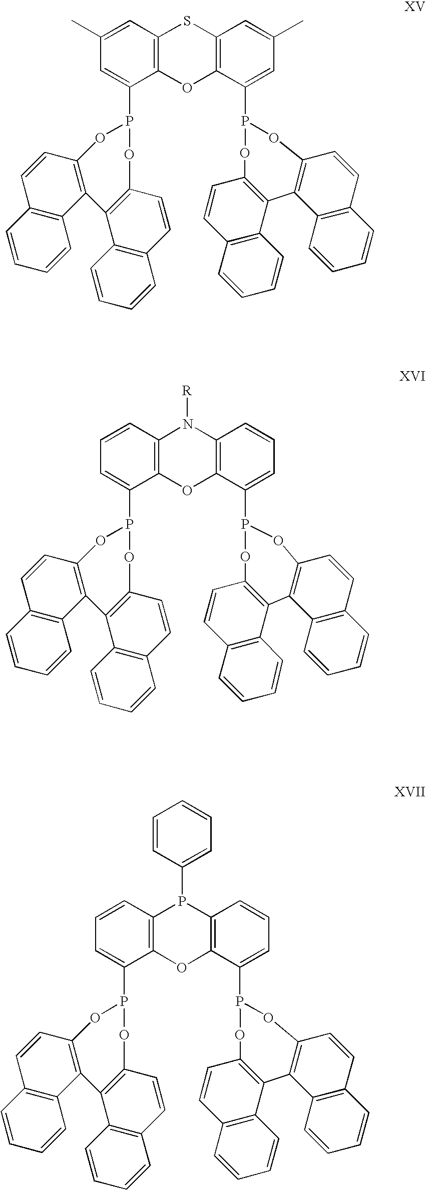

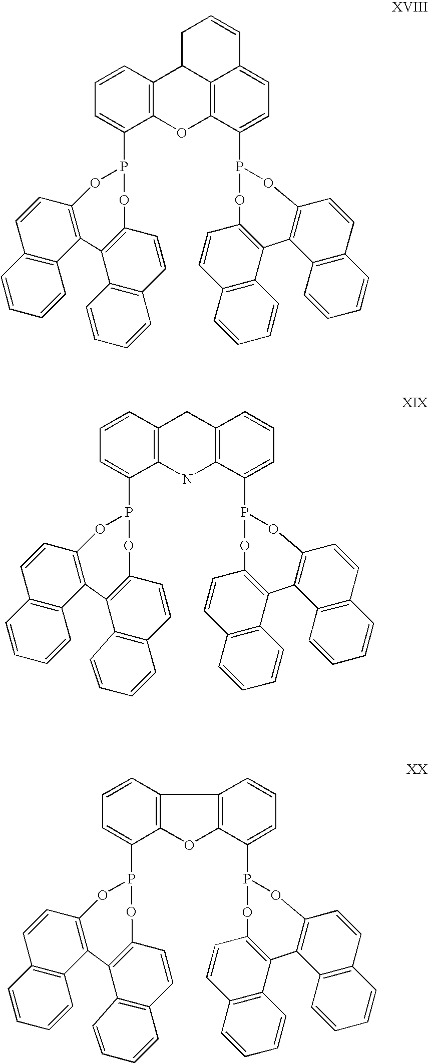

Even though the chiral assistant on the phosphorus is BINOL (A) in all diphosphonites described above, the invention is not restricted to this specific chiral diol. Octahydro-BINOL (B) can also be used in addition to many others.

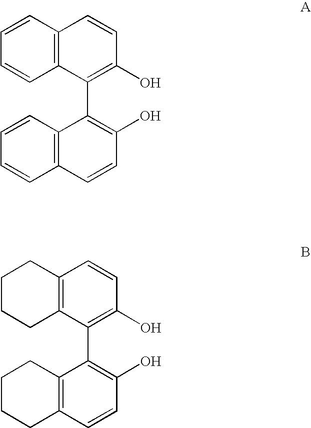

Other axial chiral diols may likewise be used; many of them have been prepared according to the literature using efficient synthesis processes, for example substituted BINOL derivatives C, substituted diphinol derivatives D with axial chirality and diols with axial chirality which contain the heterocycles according to E.

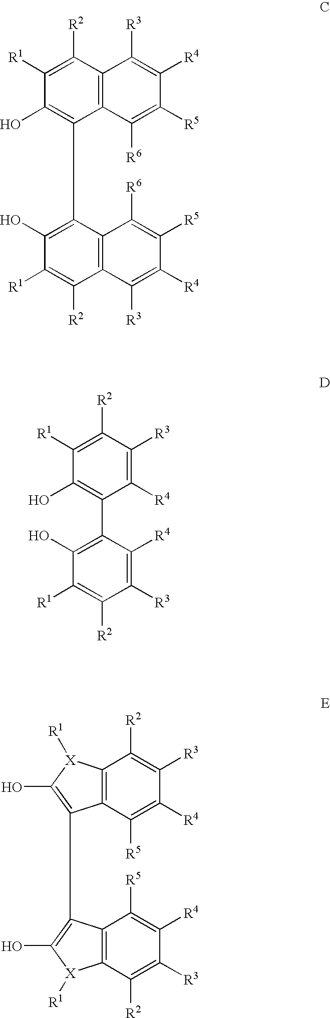

In the case of the chiral assistant C, the oxygen-containing base block consists of binaphthol A with the R¹, R², R³, R⁴, R⁵ and R⁶ radicals which may each independently be the following groups: hydrogen (H), saturated hydrocarbons, optionally functionalized and/or bridged (e.g. R¹+R²=—(CH₂)₄—), aromatic or heteroaromatic groups which may be functionalized and/or fused and are likewise cyclic radicals (for example R¹+R²=ortho-phenylene, corresponding to 4,4′-dihydroxy-5,5′-bis(phenanthryl), nonaromatic unsaturated hydrocarbons such as alkinyl groups —C≡CR which may likewise be functionalized, silyl groups such as —SiMe₃, halogen (—Cl, —Br, —F, —I), nitro (—NO₂), or nitrile (—CN) groups, or ester (—CO₂R), amide (—C(O)NRR′), amine (—NRR′), ether (—OR), sulfide (—SR) and selenide (—SeR), in which R and R′ are each hydrogen, saturated or nonaromatic unsaturated hydrocarbons which may optionally be functionalized, or aromatic radicals which may optionally be functionalized. In particular, the present invention comprises all combinations of the radicals mentioned for R¹, R², R³, R⁴, R⁵ and R⁶ including all C₁- or C₂-symmetric substitution patterns of the base structure of binaphthol. In addition, one or more carbon atoms of the binaphthol ring may also be replaced by heteroatoms such as nitrogen. Binaphthol itself (R¹═R²═R³═R⁴═R⁵═R⁶═H) (A) preferably constitutes the base block, since it is not only one of the least expensive assistants in the field of asymmetric catalysis but also because a high efficiency is achieved when diphosphonite ligands prepared with this diol are used.

In the case of the chiral diol D, the dihydroxyl base block is a functional biphenol which is stable with regard to its configuration. The stability of the configuration with regard to axial chirality is ensured when R⁴≠H (E. L. Eliel, S. H. Wilen, L. N. Mander, Stereochemistry of Organic Compounds, Wiley, New York, 1994). R¹ to R⁴ exhibit the same range of R¹ to R⁶ radicals from compound class C. Preference is given to selecting the particularly easily obtainable derivatives D where R¹═R²═H and R⁴═OCH₃ and R³═Cl (D. J. Cram, R. C. Helgeson, S. C. Peacock, L. J. Kaplan, L. A. Domeier, P. Moreau, K. Koga, J. M. Mayer, Y. Chao, M. G. Siegel, D. H. Hoffman, G. D. Y. Sogah, J. Org. Chem. 1978, 43, 1930-1946).

In the case of the chiral diols E, the dihydroxy base block is a functionalized heteroaromatic system of stable configuration, which derives from 2,2′-dihydroxy-3,3′-bis(indolyl) (X═N), 2,2′-dihydroxy-3,3′-bis(benzo[b]thiophenyl) (X═S) or 2,2″-dihydroxy-3,3′-bis(benzo[b]furanyl) (X═O). In these cases too, the substituents exhibit the same range as in D. Substituent R¹ is absent when X═O or X═S.

Chiral spiro-diols such as F (A.-G. Hu, Y. Fu, J.-H. Xie, H. Zhou, L.-X. Wang, Q.-L. Zhou, Angew. Chem. Int. Ed. 2002, 41, 2348-2350), diols G derived from paracyclophane or C1- or C2-symmetric diols with central chirality, e.g. 1,3-diols or diols of the H type, may also be used as components in the synthesis of diphosphonite ligands.

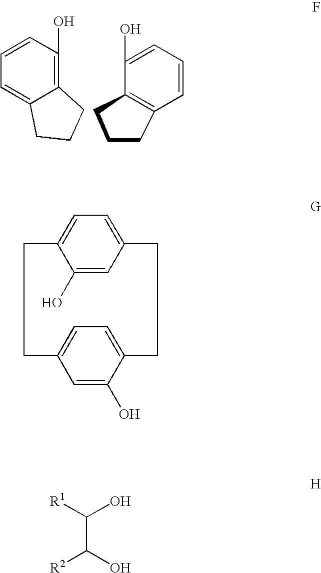

The R¹ and R² radicals in the diols H may be identical (C₂ symmetry) or different (C₁ symmetry). They may be a saturated hydrocarbon which may optionally be functionalized, as in the cases of 1,3-diol units of protected carbohydrates. Possible radicals also include aromatic or heteroaromatic groups, such as phenyl, naphthyl or pyridyl, which may themselves again be functionalized if this is desired or required. It is also possible that the radicals have ester or amide groups, such as —CO₂CH₃, —CO₂C₂H₅, —CO₂-i-C₃H₇ or —CO[N(CH₃)₂], —CO[N(C₂H₅)₂] or —CO[N(i-C₃H₇)₂], in which case the corresponding diols H are tartaric acid derivatives.

The preferred diphosphonite ligands in the Ru-catalyzed hydrogenation of ketones, β-keto esters and ketimines are those which derive from the diols A, B or D (i.e. where R¹═R²═H; R³═Cl; R⁴═OCH₃). Instead of the chiral diols, it is also possible to use chiral diamides or amino alcohols in the preparation of the chiral diphosphonites. Typical examples are I (e.g. R¹═R²Ph; R³═CH₃, PhCH₂, Ph or SO₂Ph), J (e.g. R═CH₃, Ph, CH₂Ph or SO₂Ph), K (e.g. R═CH₃, Ph, CH₂Ph or SO₂Ph) or L (e.g. R¹═Ph; R²═R³═CH₃).

As is also the case for all previous chiral ligands, all stereoisomeric forms in this case too form part of the invention.

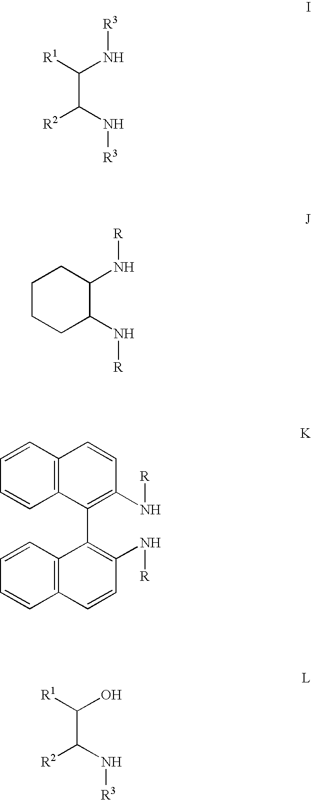

One of the most effective and therefore preferred ligands is the bisphosphonite XI or analogs thereof in which the BINOL base block has been replaced by the chiral diols B or D (e.g. R¹═R²═H; R³═Cl; R⁴═OCH₃). Since, however, no ligand can be used universally, the other diphosphonites also have to be taken into account when particular substrates are to be hydrogenated. For example, in the case of hydrogenation of β-keto esters III, the ligand X, which derives from diphenyl ether, is preferred.

The invention also encompasses novel metal complexes as catalysts, by virtue of reaction of the above-defined chiral diphosphonites with ruthenium salts, of which a great multitude are available (Encyclopedia of Inorganic Chemistry (R. B. King, Ed.), Vol. 7, Wiley, New York, 1994; Comprehensive Coordination Chemistry (G. Wilkinson, Ed.), Chapter 45, Pergamon Press, Oxford, 1987). Preference is given to using Ru(II) salts, but it is also possible to use Ru(III) salts which are reduced under the reaction conditions to Ru(II). Typical examples include those compounds such as RuX₂ (X═Cl, Br, I, SC₆H₅, AcAc, OTf), but also M, N, O, P (in which X═Cl, Br, I, SPh, OPh, OAc, AcAc or NHAc, Q (in which X═Cl, Br, I, SPh, OPh, OAc, AcAc or NHAc), R (in which X═Cl, Br, I, SPh, OPh, OAc, AcAc or NHAc), S or T. Typical Ru(III) salts include RuX₃ (X═Cl, Br, I, SPh, OPh, OAc, AcAc or NHAc).

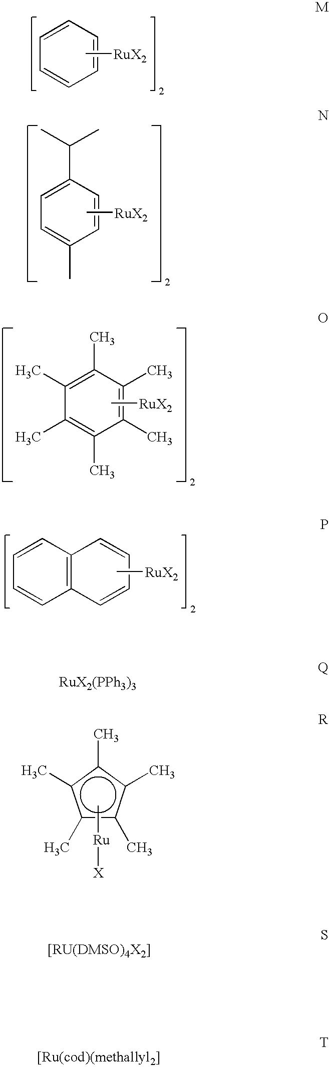

These salts, some of which are commercially available, are reacted in a simple manner with the chiral diphosphonites described to form the catalysts. The ratio of diphosphonites to Ru may be between 2:1 and 4:1, preferably 2.5:1. In general, the preferred catalysts are formed when a ratio of 2:1 is selected, but an excess of ligands may in some cases be advantageous. Some of the best catalysts for the reduction of ketones I are formed when the salts of the precursor M or N (X═Cl) are treated with the diphosphonite XI. In the case that β-keto esters III are reduced, the preferred catalysts are formed by the treatment of the salts M or N with the disphosphonite X.

The invention relates not only to complexes of the chiral diphosphonites and Ru(II) or Ru(III) salts, but also to their use as catalysts in the asymmetric hydrogenation of prochiral ketones I, keto esters III and ketimines V. The reducing agents used may be a multitude of compounds, especially in the case of hydrogenation based on the compound H₂ or in the case of transfer hydrogenation in which agents such as formic acid, alcohols, sodium dithionite or NaH₂PO₂ are used. According to the present invention, one of the most preferred variants is transfer hydrogenation using an alcohol both as reducing agent and as a solvent. A great multitude of alcohols is suitable for this purpose, and isopropanol or cyclohexanol are typically used. Isopropanol is preferred. In some embodiments of the present invention, the hydrogenation or transfer hydrogenation is performed in the presence of a base. Typical bases are NaOH, KOH, MgO, Na₂CO₃, K₂CO₃, NaF, KF, NaOCH(CH₃)₂, KOCH(CH₃)₂, NaOC(CH₃)₃ or KOC(CH₃)₃, preferred bases NaOH, KOH, NaOC(CH₃)₃ or KOC(CH₃)₃.

Typical ketones which are readily amenable to the enantioselective reduction using the catalysts and processes of the present invention are the ketones of the formulae Ia-m.

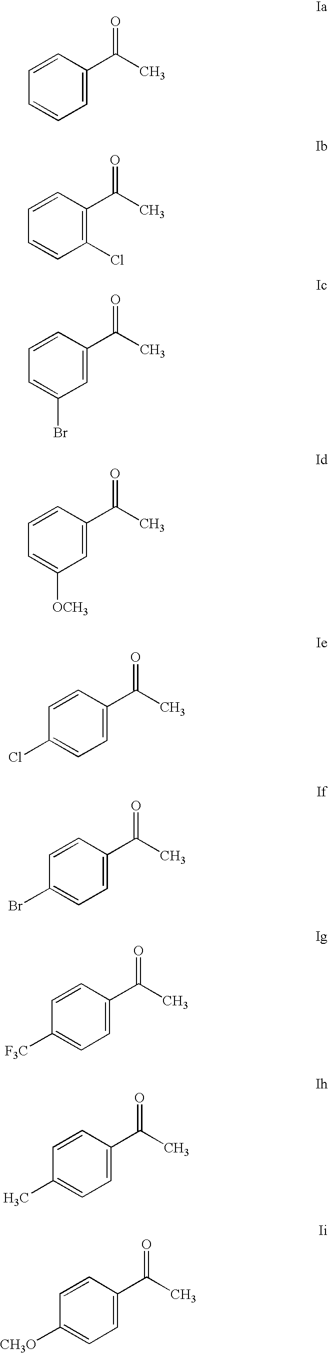

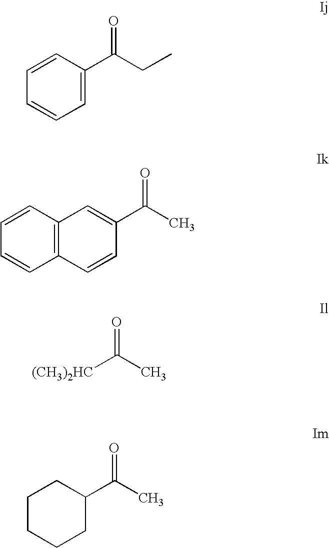

Typical β-keto esters which are subjected to the asymmetric Ru-catalyzed reduction are IIIa-e, but R¹ and R² may be varied appropriately if required.

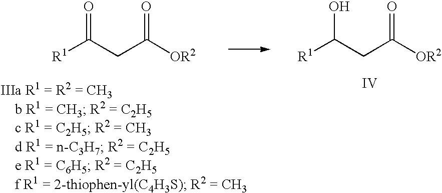

Typical corresponding substrates are those β-keto esters having a stereogenic center at the 2-position such as XXI or XXIII, which can likewise be reduced.

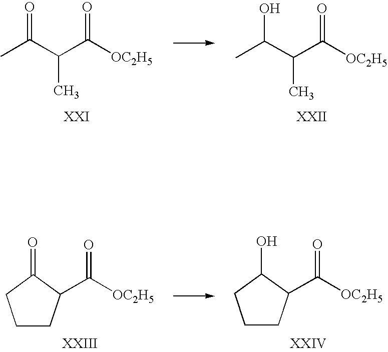

Typical prochiral ketimines which are subjected to the reduction with the Ru catalysts in the process according to the invention are those with the formulae XXVa-b or XXVII:

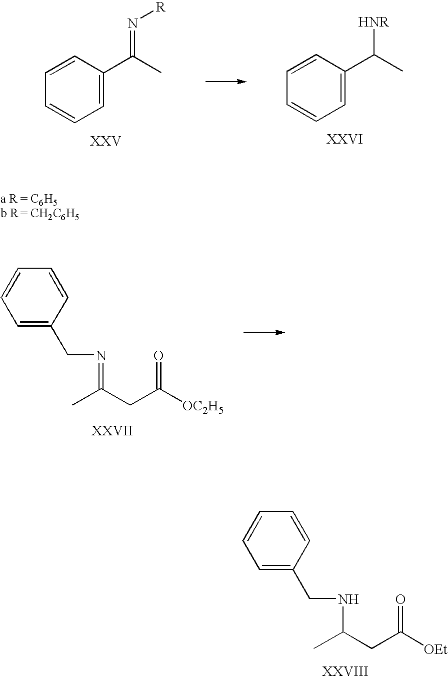

### EXAMPLES

### Typical Process for the Asymmetric Transfer Hydrogenation

[RuCl₂(p-cymene]₂ (N) (1.22 mg, 2 μmol) and a chiral diphosphonite ligand such as XI (0.010 mmol) were heated in dry isopropanol (2.5 ml) at 80° C. under argon for 1 h. Once the mixture had been cooled to room temperature, a base NaOH (0.04 mmol; 0.5 ml of a 0.08 M solution in isopropanol) or KOC(CH₃)₃ (0.04 mmol; 0.5 ml of a 0.08 M solution in isopropanol) were added, then a ketone such as acetophenone (0.4 mmol) was added. The reaction mixture was stirred at 40° C. under argon over a defined period (typically 16-96 h). Samples were taken from the reaction solution and put through a small amount of silica gel before the GC analysis to determine the conversions and the ee values by gas chromatography.

### Typical Process for the Asymmetric H₂ Hydrogenation:

[Ru(benzene)Cl₂]₂ (N) (16 mg, 0.032 mmol) and a diphosphonite (0.067 mmol) were introduced into a 25 ml Schlenk tube. The tube was purged three times with argon before dry dimethylformamide (DMF) (3 ml) was added. The resulting mixture was heated to 100° C. for 30 minutes and then cooled to 60° C. The solvent was removed under reduced pressure, and the catalyst was obtained as a pale green-yellow solid. This catalyst was dissolved in dry dichloromethane (8 ml) and distributed uniformly between 8 vials (in each case 1 ml), which had already been purged three times with argon. A ketone, such as a β-keto ester (III) (0.8 mmol), was introduced into each vessel, then in each case 3 ml of ethanol were added. These were then transferred to a high-pressure autoclave. Once it had been purged three times with H₂, the autoclave was adjusted to a pressure 60 bar with H₂, and the reactions were stirred magnetically at 60° C. over 20 h. The autoclave was subsequently cooled to room temperature and H₂ was cautiously discharged. Samples were taken from each reaction solution and put through a small amount of silica gel before the GC analysis in order to determine the conversions and ee values. The absolute configuration was determined in comparison to known compounds described in the literature.

Table 1 summarizes the results which were obtained by the above-described processes for the asymmetric transfer hydrogenation of ketones, typically using the diphosphonite XI as a chiral ligand.

TABLE 1
Typical results of an asymmetric Ru-catalyzed
transfer hydrogenation of β-keto esters using the
current process (see above) and diphosphonites XI as
ligands L* prepared with (R)-BINOL; Bu^{t} = C(CH₃)₃.

| | | | | | | | Con- |
|---|---|---|---|---|---|---|---|
| | | | | | Con- | | figuration |
| | | | | Time | version | ee | of the |
| No. | Ketone | Base | L*/Ru | (h) | (%) | (%) | product |
| 1 | Ia | KOBu^{t} | 4 | 28 | 91 | 97 | R |
| 2 | Ia | NaOH | 2.5 | 20 | 88 | 97 | R |
| 3 | Ia | NaOH | 2.5 | 40 | 93 | 98 | R |
| 4 | Ib | KOBu^{t} | 4 | 30 | 50 | 98 | R |
| 5 | Ib | NaOH | 2.5 | 26 | 83 | 99 | R |
| 6 | Ib | NaOH | 2.5 | 40 | 90 | 99 | R |
| 7 | Ic | KOBu^{t} | 4 | 28 | 100 | 95 | R |
| 8 | Ic | NaOH | 2.5 | 16 | 100 | 96 | R |
| 9 | Id | NaOH | 2.5 | 40 | 63 | 93 | R |
| 10 | Id | NaOH | 2.5 | 96 | 91 | 93 | R |
| 11 | Ie | KOBu^{t} | 4 | 22 | 96 | 96 | R |
| 12 | Ie | NaOH | 2.5 | 16 | 98 | 95 | R |
| 13 | If | NaOH | 2.5 | 26 | 98 | 95 | R |
| 14 | Ig | NaOH | 2.5 | 16 | 100 | 97 | R |
| 15 | Ih | KOBu^{t} | 4 | 30 | 67 | 95 | R |
| 16 | Ih | NaOH | 2.5 | 26 | 65 | 94 | R |
| 17 | Ii | KOBu^{t} | 4 | 30 | 50 | 89 | R |
| 18 | Ij | NaOH | 2.5 | 26 | 65 | 93 | R |
| 19 | Ik | NaOH | 2.5 | 22 | 56 | 93 | R |
| 20 | Il | NaOH | 2.5 | 26 | 98 | 98 | S |
| 21 | Im | NaOH | 2.5 | 26 | 96 | 99 | R |

The results of an asymmetric H₂ hydrogenation of β-keto esters III are compiled in Table 2.

TABLE 2
Results of an asymmetric Ru-catalyzed H₂
reduction of β-keto esters using the current process
(see above) and diphosphonite X as a ligand prepared
with (S)-BINOL.

| β-Keto | Conversion | | Configuration of the |
|---|---|---|---|
| ester | (%) | ee (%) | product |
| IIIa | 100 | 93 | S |
| IIIb | 100 | 95 | S |
| IIIc | 100 | 95 | S |
| IIId | 100 | 97 | S |
| IIIe | 100 | 95 | R |
| XXI | 100 | 95/95^{a)} | Anti-diastereomer: (2S, 3S) |
| XXIII | 100 | 99^{b)} | Anti-diastereomer: (1S, 2S) |

^{a)}Diastereomeric ratio is 1:1, in each case 95% ee;
^{b)}Only one diastereomer (96:4).

1. A chiral ruthenium complex prepared by reacting a ruthenium salt with a chiral diphosphonite.

2. A ruthenium complex as claimed in claim 1, in which the diphosphonite is derived from a chiral diol of the structure shown below:

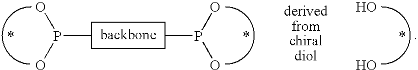

3. A ruthenium complex as claimed in claim 2, in which the diol in has the formula C, D or E with R¹, R², R³, R⁴, R⁵ and R⁶ groups, each of which is independently: hydrogen, saturated carbon chains which may each be functionalized and/or bridged, aromatic or heteroaromatic radicals which may each be functionalized and/or bridged, nonaromatic unsaturated carbon chains which may each be functionalized, silyl groups, halogen, nitro, nitrile, ester, amide, amine, ether or thioether radicals;

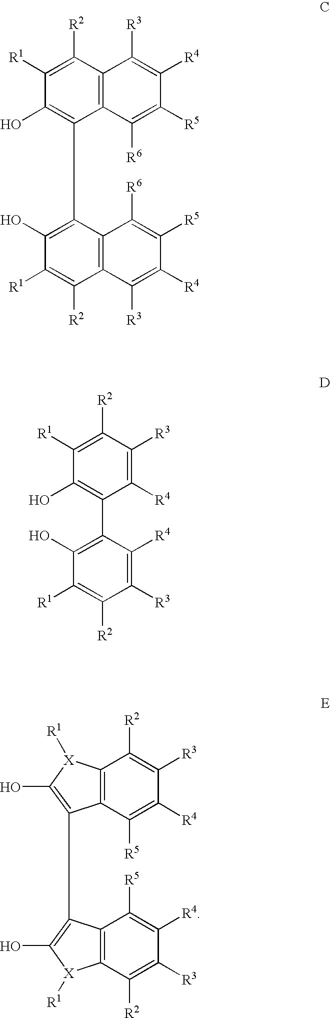

4. A ruthenium complex as claimed in claim 3, in which the diol has the formula A, B or DI:

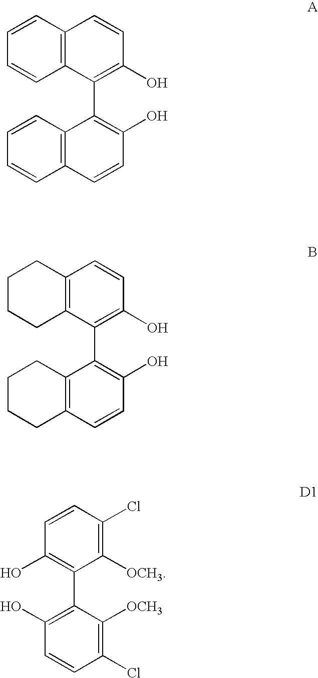

5. A ruthenium complex as claimed in claim 2, in which the diol has the formula F or has the formula G or H, in which R¹═R² or R¹≠R², where these radicals are methyl, ethyl, propyl, butyl, phenyl, naphthyl, oxyl or carboxamido:

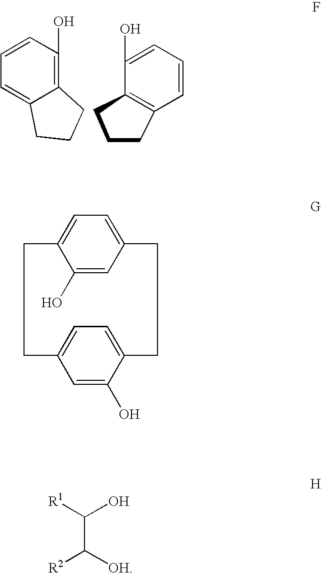

6. A ruthenium complex as claimed in claim 1, in which a chiral radical on the phosphorus is derived from a chiral diamine.

7. A ruthenium complex as claimed in claim 6, in which the chiral diamine has the formula I, J or K, where the R, R¹, R² and R³ radicals are each saturated C₁-C₁₀ carbon groups, aryl groups, sulfonyl derivatives, carboxyl derivatives or carboxamido derivatives:

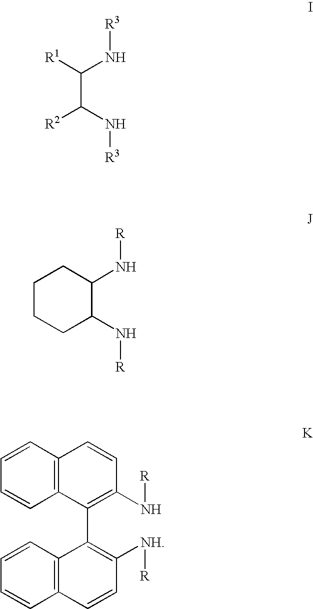

8. A ruthenium complex as claimed in claim 1, in which a chiral radical on the phosphorus derives from a chiral amino alcohol L:

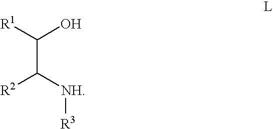

9. A ruthenium complex as claimed in claim 8, in which the chiral amino alcohol L has the formula L1 or L2:

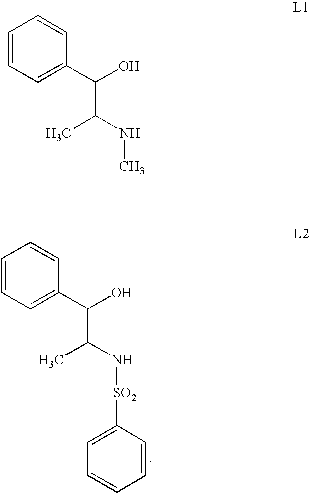

10. A ruthenium complex as claimed in claim 8, which comprises an achiral backbone, excluding a backbone based on ferrocene.

11. A ruthenium complex as claimed in claim 10, in which the achiral backbone derives from one of the U1-U15 radicals:

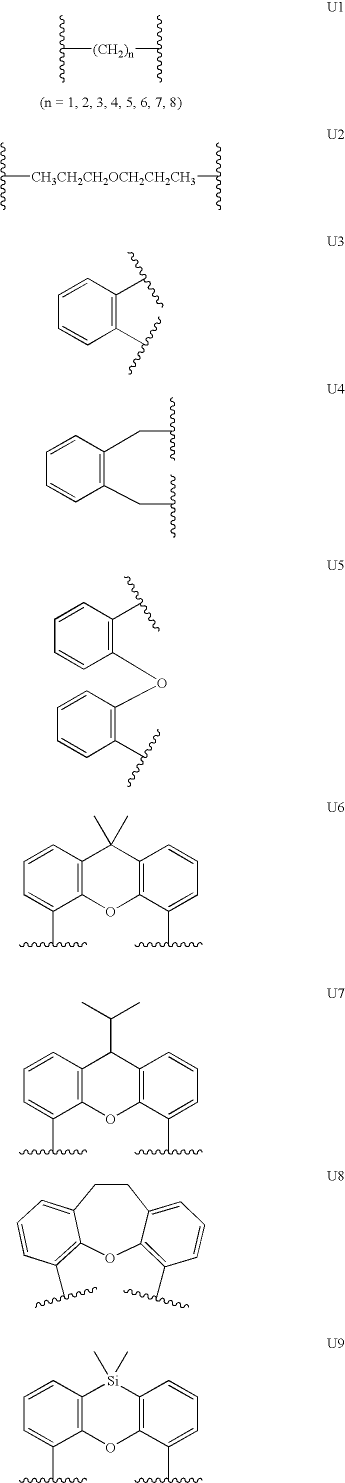

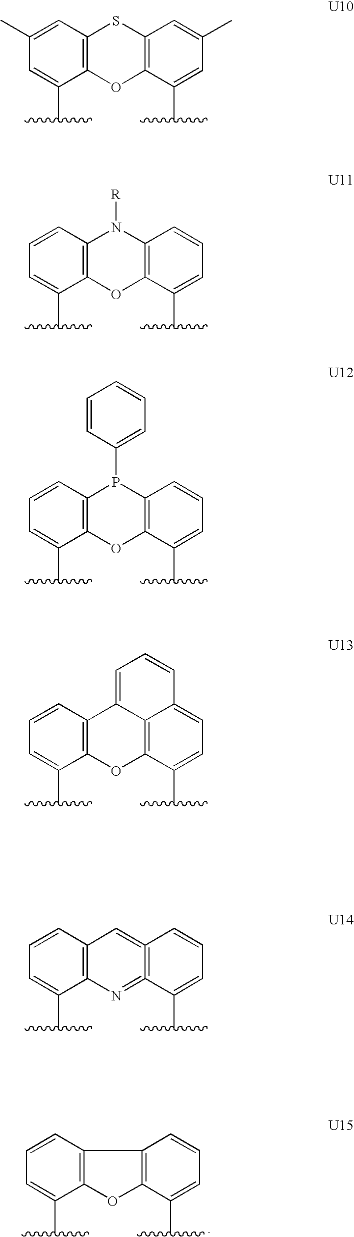

12. A ruthenium complex as claimed in claim 1, which is prepared by a process comprising reacting Ru(II) salts.

13. A ruthenium complex as claimed in claim 12, in which the Ru(II) salts have the formula M, N, O, P, Q, R, S or T: 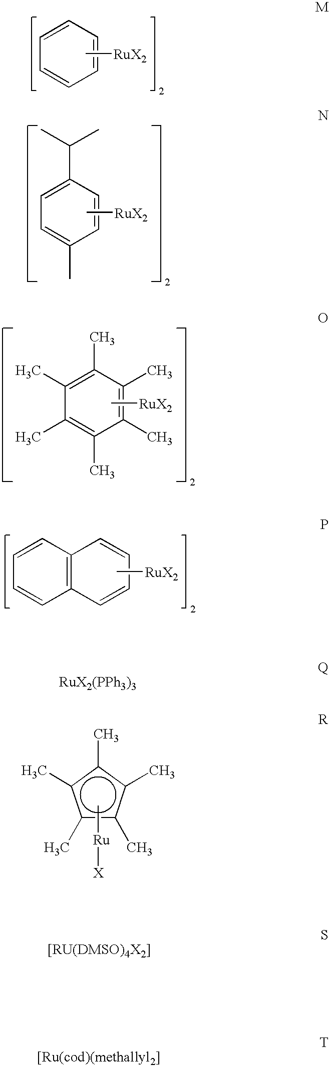
in which X═Cl, Br, I, OAc, OC₆H₅, SC₆H₅, AcAc₁, OTf, or NHAc.

14. A ruthenium complex as claimed in claim 1, which is prepared by a process comprising reacting Ru(III) salts.

15. A ruthenium complex as claimed in claim 14, in which the Ru(III) salt is RuX₃ (X═Cl, Br, I, SC₆H₅, AcAc, OTf).

16. A process comprising asymmetric reduction of prochiral ketones, β-keto esters or ketimines in the presence of a ruthenium complex as claimed in claim 1.

17. The process as claimed in claim 16, in which H₂ is used as a reducing agent.

18. The process as claimed in claim 16, in which an alcohol, formic acid, sodium formate or ammonium formate or an inorganic Na₂S₂O₄ or NaH₂PO₂ reducing agent is used in a transfer hydrogenation.

19. The process as claimed in claim 18, in which isopropanol or cyclohexanol is used as a reducing agent.

20. The process as claimed in claim 16, which further comprises adding a base to the reaction mixture.

21. The process as claimed in claim 20, in which the base is NaOH, KOH, MgO, Na₂CO₃, K₂CO₃, NaF, KF, NaOCH(CH₃)₂, KOCH(CH₃)₂, NaOC(CH₃)₃ or KOC(CH₃)₃.
